(19) United States
(12) Patent Application Publication
(10) Pub. No.: US 2011/0195442 A1
(43) Pub. Date: Aug. 11, 2011

(54) STERILITY INDICATING BIOLOGICAL COMPOSITIONS, ARTICLES AND METHODS

(76) Inventors: Sailaja Chandrapati, Woodbury, MN (US); Heather M. Webb, Woodbury, MN (US); Ai-Ping Wei, Woodbury, MN (US)

(21) Appl. No.: 13/123,824

(22) PCT Filed: Oct. 15, 2009
(86) PCT No.: PCT/US2009/060805

§ 371 Date: Apr. 12, 2011

Related U.S. Application Data
(60) Provisional application No. 61/196,414, filed on Oct. 17, 2008.

(51) Int. Cl. C12Q 1/37

(52) U.S. Cl. 435/23

(57) ABSTRACT

A sterility indicating composition comprising a plurality of sterilization process resistant spores which contain an active protease during germination and initial outgrowth of the spores; and a germination medium comprising at least one labeled protease substrate and at least one nutrient for germination of the spores; wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores, a sterilization process indicator comprising the composition, and a method of determining the effectiveness of a sterilization process using the composition and indicator are disclosed.

### CROSS REFERENCE TO RELATED APPLICATIONS

This application claims the benefit of U.S. Provisional Application No. 61/196,414, filed Oct. 17, 2008, which is incorporated herein by reference in its entirety.

### BACKGROUND

Primarily in the health care industry, but also in many other industrial applications, it is necessary to monitor the effectiveness of processes used to sterilize equipment such as medical devices, instruments and other non-disposable articles. In these settings, sterilization is generally defined as the process of completely destroying all viable microorganisms including structures such as viruses and spores. As a standard practice, hospitals include a sterility indicator with a batch of articles to assay the lethality of the sterilization process. Both biological and chemical sterility indicators have been used.

A standard type of biological sterility indicator includes a known quantity of test microorganisms, for example Bacillus stearothermophilus or Bacillus subtilis spores, which are many times more resistant to a sterilization process than most contaminating organisms. After the indicator is exposed to the sterilization process, the spores are incubated in nutrient medium to determine whether any of the spores survived the sterilization process, with spore growth indicating that the sterilization process was insufficient to destroy all of the microorganisms. In another example, after being subjected to a sterilization process, the activity of an enzyme, which can be correlated with spore viability, is determined. Although advances have been made; the time period for determining this with certainty can be undesirably long.

Available chemical sterility indicators can be read immediately at the end of the sterilization process. However, the results indicate only that a particular condition was present, such as the presence of a particular chemical or a temperature for a certain period of time.

It is generally considered that the response of living organisms to all conditions actually present is a more direct and reliable test for how effective a sterilization process is in achieving sterilization. Accordingly, there is a continuing need for biological sterility indicators which can indicate the effectiveness of a sterilization process without an excessive delay after completion of the sterilization process.

### SUMMARY

The present invention provides a biological sterility indicator composition, a sterility indicator including the composition, and a method of determining the effectiveness of a sterilization process using the indicator. The composition includes sterilization process resistant spores which contain an active protease during germination and initial outgrowth of the spores, for example, spores that have survived a sterilization process. In certain embodiments, the active protease, if present, can be detected during and/or after a short incubation time. The active protease is detected in the presence of a labeled protease substrate. The substrate is labeled with one or more dye groups, at least one of which is detectably changed when a peptide portion of the substrate is cleaved by the active protease. The labeled protease substrate is stable at least at a temperature for incubating the spores, and in certain embodiments, preferably at a sterilization temperature.

Accordingly, in one embodiment, there is provided a sterility indicating composition comprising:

a plurality of sterilization process resistant spores which contain an active protease during germination and initial outgrowth of the spores;

a germination medium comprising at least one labeled protease substrate and at least one nutrient for germination of the spores;

wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and

wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores.

In another embodiment, there is provided a sterilization process indicator comprising:

a carrier supporting a plurality of sterilization process resistant spores which contain an active protease during germination and initial outgrowth of the spores;

a container impermeable to microorganisms and impermeable to a sterilant, the container containing a germination medium comprising at least one labeled protease substrate and at least one nutrient for germination of the spores;

wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and

wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and

wherein the carrier is adjacent to the container and separate from the germination medium.

In a further embodiment, there is provided a method of determining the effectiveness of a sterilization process, the method comprising:

providing a sterilization process indicator comprising:
a carrier supporting a plurality of sterilization process resistant spores which contain an active protease during germination and initial outgrowth of the spores;
a container impermeable to microorganisms and impermeable to a sterilant, the container containing a germination medium comprising at least one labeled protease substrate and at least one nutrient for germination of the spores;
   wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and
   wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and
wherein the carrier is adjacent to the container and separate from the germination medium;

positioning the sterilization process indicator in a sterilization chamber;

exposing the sterilization process indicator to a sterilant;

combining the plurality of sterilization process resistant spores and the germination medium;

incubating the spores with the germination medium;

and

measuring the detectable change, if present.

### Definitions

The terms “contain an active protease” and “active protease contained by” refer to an active protease within the spores, in the spore coat, and/or on the spores.

The term “essentially free of any active protease” refers to a sufficiently low level of any competing active protease, such that the detectable change resulting from the active protease from the spores can be measured and/or a detectable change resulting from the competing active protease is not more than 10 percent greater than when no competing active protease is present.

The term “essentially free of any protease substrate” refers to a sufficiently low level of any competing protease substrate, such that the detectable change is not decreased by more than 10 percent and/or any competing protease substrate does not substantially delay the time required for the detectable change to occur compared to when no competing protease substrate is present. For certain embodiments, a substantial delay is more than a 2 fold increase in the time required for the detectable change to occur.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., 550 to 600 nm includes 550, 551, 575, 583, 592, 600, etc.).

The above summary of the present invention is not intended to describe each disclosed embodiment or every implementation of the present invention. The description that follows more particularly exemplifies illustrative embodiments.

### BRIEF DESCRIPTIONS OF THE FIGURE

FIG. 1 is a cross-sectional view of one embodiment of a sterility indicator of the present invention, with cap 26 not present.

FIG. 2 is an exploded perspective view of the sterility indicator of FIG. 1, with cap 26 included.

### DETAILED DESCRIPTION OF ILLUSTRATIVE EMBODIMENTS OF THE INVENTION

As indicated above, biological sterility indicator compositions are now provided which can detect viable spores during germination and initial outgrowth of the spores, using a dye-labeled protease substrate which is stable at least at temperatures for incubating the spores, for example, for germination and initial outgrowth of the spores. For certain embodiments, preferably such temperatures are up to at least 37° C., more preferably up to at least 50° C., even more preferably up to at least 60° C. The spores are incubated with the germination medium, which includes the labeled protease substrate.

Significant sensitivity during initial stages of germination and outgrowth of viable spores is provided, at least in part, by the medium portion of the composition being essentially free of any active protease (other than the active protease contained by the spores when the germination medium and the spores are contacted with each other). Any baseline level of a detectable change in the dye-label resulting from the presence of an active protease not contained by the viable spores is, therefore, minimized. The composition is also essentially free of any protease substrate, other than that generated in situ or de Novo within the spores, which can compete with the labeled protease substrate. This makes any active protease contained by viable spores available to act on the labeled protease substrate and thereby avoid a loss in the level of the detectable change and/or a time delay in the occurrence of the detectable change.

The biological sterility indicator compositions can be used advantageously in biological sterility indicators, such as those described herein. The compositions and indicators containing the compositions are useful in determining the effectiveness of a sterilization process, such as in the method, described herein, of determining the effectiveness of a sterilization process.

A number of sterilization processes are presently known and in use, including, for example, exposure to steam, dry heat, gaseous or liquid agents such as ethylene oxide, hydrogen peroxide, and peracetic acid, and radiation. The plurality of sterilization process resistant spores are selected according to the sterilization process to be used. For example, for a steam sterilization process, Gb. stearothermophilus (formerly Bacillus stearothermophilus) may be used. In another example, for an ethylene oxide sterilization process, B. atrophaeus (formerly B. subtilis) may be used. For certain embodiments, including any one of the above composition, indicator, and method embodiments, the plurality of sterilization process resistant spores is selected from the group consisting of Gb. stearothermophilus, B. atrophaeus, B. megaterium, Clostridium sporogenes, Bacillus coagulans, and a combination thereof. For certain of these embodiments, the plurality of sterilization process resistant spores is selected from the group consisting of Gb. stearothermophilus, B. atrophaeus, B. megaterium, and a combination thereof. It has been estimated that about 10 to 20 percent of the proteins in dormant spores of the Bacillus species are small, acid-soluble proteins, which bind to DNA and increase resistance of the DNA to various damaging agents. During spore germination, degradation of these proteins is initiated by a sequence-specific endoprotease (GPR), which is activated during the germination of the spores. See Y. Carrillo-Martinez and P. Setlow, J. Bacteriology, 176, 5357-5363 (1994).

By way of example only, the present disclosure describes the microorganisms used in the biological sterilization indicator as being “spores;” however, it should be understood that the type of microorganism (e.g., spore) used in a particular embodiment of the biological sterilization indicator is selected for being highly resistant to the particular sterilization process contemplated. Accordingly, different embodiments of the present disclosure may use different microorganisms, depending on the sterilization process for which the particular embodiment is intended.

After exposure to a sterilization process which is sub-lethal to the plurality of sterilization resistant spores, the protease remains sufficiently active to react with the labeled protease substrate when incubated with the germination medium. For certain embodiments, including any one of the above composition, indicator, and method embodiments, the active protease has no more than a background level of activity when subjected to a sterilization process which is just sufficient to decrease a population of at least 1×10⁵ spores to zero, as measured by lack of outgrowth of the spores; and has a level of activity greater than the background level of activity when subjected to a sterilization process sufficient to decrease the population of at least 1×10⁵ spores by at least one log but to a population greater than zero; wherein the level of activity is measured by reacting an effective amount of the at least one labeled protease substrate with the active protease to produce the detectable change in at least one of the one or more dye groups, and measuring the detectable change. Sources of background activity include, for example, auto-hydrolysis or other degradation routes of the labeled substrate, in which signal generated is not due to germination of the plurality of the sterilization resistant spores. For certain embodiments, an effective amount of the at least one labeled protease substrate is an amount sufficient to react with any active protease present to produce a measurable detectable change. Such a detectable change is measurable within 8 hours, preferably within 1 hour, more preferably within 30 minutes, even more preferably within 15 minutes. For certain embodiments, including any one of the above composition, indicator, and method embodiments, the active protease is germination specific protease. This protease is also known as GPR.

The labeled protease substrate is stable at a temperature, for example, an incubation temperature and/or a sterilization temperature, such that, during or after exposure to the temperature, in the presence of active protease the labeled protease substrate undergoes a detectable change which can be observed or measured. For certain embodiments, including any one of the above composition, indicator, and method embodiments, preferably the labeled protease substrate is stable at a temperature (for example an incubation temperature) up to at least 37° C. For certain of these embodiments, more preferably the labeled protease substrate is stable at a temperature up to at least 50° C. For certain of these embodiments, even more preferably the labeled protease substrate is stable at a temperature up to at least 60° C.

In at least some of the sterilization processes in use, an elevated temperature, for example, 50° C., 100° C., 121° C., 132° C., 134° C., or the like, is included or may be encountered in the process. Accordingly, for certain embodiments, including any one of the above composition, indicator, and method embodiments, the labeled protease substrate is stable at a sterilization temperature.

For certain embodiments, including any one of the above composition, indicator, and method embodiments, the labeled protease substrate is stable at a temperature up to at least 121° C. For certain of these embodiments, the labeled protease substrate is stable at a temperature up to at least 132° C. For certain of these embodiments, the labeled protease substrate is stable at a temperature up to at least 134° C.

The compositions, indicators, and methods described herein include at least one labeled protease substrate. This substrate comprises a peptide which is cleaved by the active protease when contacted by the active protease. For certain embodiments, including any one of the above embodiments, the labeled protease substrate is a labeled protein, wherein the protein is cleaved by the active protease. Sites in the protein occupied by the above peptide are cleaved by the active protease. For certain of these embodiments, the labeled protease substrate is selected from the group consisting of a labeled casein, a labeled collagen, a labeled gelatin, a labeled fibrinogen, and a combination thereof. Each of these substrates is essentially free of any active protease. For certain of these embodiments, the labeled protease substrate is a labeled casein.

For certain embodiments, including any one of the above embodiments, the peptide contains an amino acid sequence selected from the group consisting of AA₁-Glu-AA₂-Ala-AA₃-Glu-Phe, AA₄-Glu-Phe-AA₅-AA₆-Glu-AA₇, and a combination thereof; wherein AA₁ is Tyr, Leu, Phe, or Glu; AA₂ is Ile or Val; AA₃ is Ser, Gln, or Asn; AA₄ is Thr, Ala, Glu, or Gln; AA₅ is Ala, Gly, or Ser; AA₆ is Ser, Thr, or Asn; and AA₇ is Thr or Phe. Peptides with these amino acid sequence cleavage sites are readily cleaved by the active protease, such as germination specific protease, produced during germination and initial outgrowth of the spores.

The peptide is labeled with one or more dye groups, at least one of which undergoes a detectable change when the peptide is cleaved. Dye groups which can be used for this purpose are known and described, for example, in U.S. Pat. No. 7,256,012 (Wei et al.) and U.S. Pat. No. 7,410,769 (Burroughs-Tencza). Some non-limiting examples of dyes which may be used as labels include fluorescein, tetramethylrhodamine, rhodamine B, lissamine, rhodamine X, Texas Red, cyanine dyes, bodipy dyes, alexa dyes, and other fluorescent dyes commonly available from Invitrogen Corp (Carlsbad, Calif.). Other dyes known in to those skilled in the art may also be used.

Briefly, at least one dye group is attached to the protease substrate. The dye group may have a visibly observable or optically measurable characteristic. For example, the dye group may have an observable color; the dye group may have an absorbance maximum at a particular wavelength, a level of absorbance at a particular wavelength or wavelength range, certain color coordinate values, or the like, which can be measured by spectrophotometric and/or colorimetric means; or the dye group may emit light at a particular wavelength or wavelength range or at a particular intensity, which can be measured by fluorometric or luminometric means. For certain embodiments, when the protease substrate is intact, the dye group is in sufficient proximity to at least one second group for the second group to modulate the fluorescence and/or absorbance spectrum of the dye group. The second group may be, for example, another dye group, a fluorescent energy transfer acceptor, a chromophoric light absorbing compound, or a quencher. Modulation of the fluorescence or light absorbance of the dye group may occur by various mechanisms including, for example, dye dimerization and/or an energy transfer mechanism which may include nonradiative energy transfer, radiative energy transfer, intramolecular resonance energy transfer, and/or the like. When the substrate is cleaved by the active protease, the modulation is reduced or eliminated, causing a detectable change in the dye group, for example, an optical change such as a change in fluorescence intensity, a change in color, a change in intensity of a color, and/or the like.

For certain embodiments, including any one of the above composition, indicator, and method embodiments, preferably the detectable change is a change in fluorescence intensity. For certain of these embodiments, the fluorescence has a wavelength of 500 nm to 600 nm, preferably 550 nm to 600 nm. This wavelength range is advantageous in that such fluorescence may not be obscured or may be significantly less obscured by absorbance or fluorescence of the germination medium, which often occurs at shorter wavelengths, such as wavelengths less than 500 nm or less than 400 nm.

The germination medium and the sterilization process resistant spores are kept separate but in close proximity to each other for ease of combining the medium with the spores when desired, for example, after exposure to a sterilization process and incubating to determine whether or not any viable spores are present, or after exposure to a sterilization process but without incubating to determine a baseline or background of the detectable change. For example, a background level of absorbance or fluorescence at a particular wavelength may be measured. Accordingly, for certain embodiments, including any one of the above composition, indicator, and method embodiments, the plurality of sterilization process resistant spores and the germination medium are separate from each other and adjacent each other.

For certain embodiments, including any one of the above composition, indicator, and method embodiments, the germination medium is an aqueous solution or suspension. The medium contains at least one labeled protease substrate and at least one nutrient for germination of the spores, which can be dissolved or suspended in the aqueous medium. The concentration of substrate in the medium is dependent on the labeled protease substrate used and the rate at which the active protease cleaves the substrate, with more being desired when the detectable change is less readily observed or measured or the rate is relatively low. Preferably, the amount of substrate is sufficient to react within a short time with any active protease present after exposure of the spores to a sterilization process. A short time is less than 8 hours, preferably less than 1 hour, more preferably less than 30 minutes, and even more preferably less than 15 minutes. For certain of these embodiments, the concentration of the labeled protease substrate is at least 0.01 mg/mL. For certain of these embodiments, the concentration of the labeled protease substrate is at least 0.1 mg/mL.

The medium contains at least one nutrient that induces germination and initial outgrowth of the spores, if viable, with the simultaneous production of active protease. The nutrient includes one or more sugars, for example, glucose, fructose, cellibiose, or the like. The nutrient may also include a salt such as potassium chloride, calcium chloride, or the like. For certain embodiments, the nutrient further includes at least one amino acid, for example, at least one of methionine, phenylalanine, and tryptophan. The germination medium may also include one or more other materials with the nutrient. For example, lysozyme, which can help induce spore germination and release of active protease, may be included. The quantities of such nutrients and materials as well as other nutrients known in the art for inducing germination and initial outgrowth of the sterilization process resistant spores may be used. Medium components and concentrations are known and described, for example, in WO 99/05310 (Tautvydas) and Zechman et al., J. Food Sci., 56, 5, pages 1408-14011 (1991) (also known as Zechman and Pflug, 1991).

In one alternative, for certain embodiments, the germination medium is in a dry form. The at least one labeled protease substrate and at least one nutrient can be dried separately or together to form a film(s) or a layer(s) on a support film or on a carrier material in a desired shape, or compounded separately or together as dry solids to form a tablet(s), caplet(s), or capsule(s). Any of these forms can be kept adjacent the spores, and water or an aqueous buffer can be added at an appropriate time to incubate the spores with the resulting medium suspension or solution. When re-suspended or dissolved, the resulting medium can be a liquid or a gel. Additional embodiments of a medium in a dry form, which can be used in the composition, indicator, and method embodiments described herein are described in copending U.S. Patent Application Ser. No. 61/196,438 (Chandrapati et al.) filed Oct. 17, 2008, entitled Biological Sterilization Indicator, System, and Methods of Using Same.

The germination medium may also include a buffer to hold the pH in a desired range. In one example, the pH may be kept within a particular range to control the absorbance and emission maxima of the at least one dye. For example, a resorufin dye-label (N-(resorufin-4-carbonyl)piperidine-4-carbonic acid N-hydroxysuccinimide ester, available from Roche Molecular Biochemicals, Mannheim, Germany) has absorbance and emission maxima at 467 nm and 559 nm, respectively, at a pH less than 7, but at 574 nm and 584 nm, respectively, at a pH greater than 7. For certain embodiments, including any one of the above composition, indicator, and method embodiments, the labeled protease substrate is a labeled casein, wherein the label is the above resorufin dye-label.

When incubating the spores with the germination medium, an incubation temperature above room temperature may be used. For certain embodiments, the incubation temperature is at least 37° C. For certain embodiments, preferably the incubation temperature is at least 50° C. or up to at least 60° C. For certain embodiments, the incubation temperature is 50 to 60° C.

As indicated above, the sterilization process indicator provided herein comprises a carrier supporting a plurality of sterilization process resistant spores which contain an active protease during germination and initial outgrowth of the spores. For certain embodiments, the carrier is a sheet material such as paper, woven cloth, nonwoven cloth, plastic, a polymeric material (e.g., polypropylene, polyethylene, polystyrene, and the like), a microporous polymeric material, metal foil, glass, porcelain, ceramic, or the like, or a combination thereof. For certain embodiments, the sheet material is water-absorbent or can be wetted to aid in quickly bringing the germination medium in intimate contact with the spores at the appropriate time.

The sterilization process indicator also comprises a container, which contains the germination medium without allowing the sterilant or any microorganism to enter the container. As indicated above, the carrier is adjacent to the container for ease of contacting the spores supported by the carrier with the germination medium when incubation is to be initiated. The container can be readily opened to contact the spores with the medium by expelling a plug, crushing or puncturing the container, or the like. The container can be equipped with a plug, or at least a portion of the container can be a breakable material, such as glass or other material, which can be breached by physical pressure but sufficiently tough to remain intact during manufacturing, storage, shipping, and sterilization conditions.

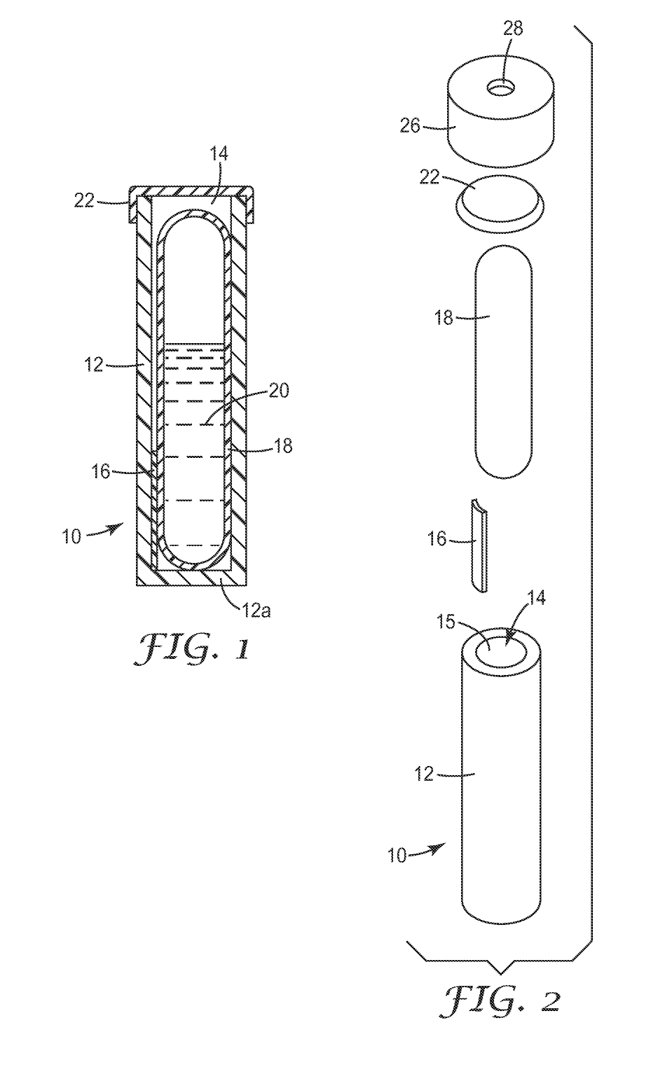

Known biological sterilization process indicator constructions such as those described in U.S. Pat. No. 5,073,488 (Matner et al.) may be used with the above described compositions. Other indicator constructions may also be used, such as those described in U.S. Patent Application Ser. No. 61/196,438 (Chandrapati et al.) filed Oct. 17, 2008, entitled Biological Sterilization Indicator, System, and Methods of Using Same. One embodiment of the sterilization process indicator described herein is shown in FIGS. 1 and 2. The indicator includes a housing 10 having an open chamber 14 defined by gas and liquid impermeable walls 12. Alternative embodiments for these structures are shown in U.S. Patent Application Ser. No. 61/196,438 (Chandrapati et al.) filed Oct. 17, 2008, entitled Biological Sterilization Indicator, System, and Methods of Using Same. The housing is shown as a circular tube, but other known configurations can be used. The walls are preferably transparent or translucent to the extent that a color change within the chamber can be visually observed, an absorbance at a particular wavelength measured, or a fluorescence intensity at a particular wavelength measured. Suitable materials for the walls may include glass, polycarbonate, polypropylene, polyester, and the like. For certain embodiments, at least one wall of the housing transmits at least 90% of incident light within a wavelength range of at least 550 to 600 nm, preferably at least 500 to 600 nm. The chamber contains a carrier 16, for example, a paper, glass, or polymeric sheet, with a predetermined number of viable sterilization process resistant spores supported by the carrier.

Container 18, which holds germination medium 20, is shown within chamber 14. Alternatively, container 18 can be positioned outside of and adjacent chamber 14. Container 18, which is sealed, can be a breakable ampoule, but could alternatively be a container equipped with a plug, or other mechanism which when activated allows germination medium 20 to contact carrier 16 and the spores supported thereon. Container 18 is shown as an elongated ampoule, but other known configurations can be used as well.

Carrier 16 is shown between container 18 and wall 12 of housing 10 for ease of determining the detectable change through wall 12. Alternatively, a portion of wall 12 may be used as carrier 16. Other placements of carrier 16 may be used, for example, placement adjacent bottom wall 12A may be used. Carrier 16 may be shaped to fit this placement based upon the shape of housing 10, or bottom wall 12A may itself be used as carrier 16. For certain embodiments, carrier 16 transmits at least 90% of incident light within a wavelength range of at least 550 to 600 nm, preferably at least 500 to 600 nm. Opening 15 to chamber 14 is provided with a gas-transmissive, microorganism-impermeable closure member 22, which may be adhered to housing 10 by an adhesive, a heat seal, or the like. Alternatively, closure member 22 may be held on to opening 15 with a cap 26 having an aperture 28. During exposure to a sterilant, the sterilant passes through the closure member 22, enters chamber 14, and contacts the spores on carrier 16. Alternative embodiments for these structures are shown in U.S. Patent Application Ser. No. 61/196,438 (Chandrapati et al.) filed Oct. 17, 2008, entitled Biological Sterilization Indicator, System, and Methods of Using Same.

The method provided herein of determining the effectiveness of a sterilization process includes providing any one of the above embodiments of a sterilization process indicator and positioning the sterilization process indicator in a sterilization chamber. Sterilizers, many of which are commercially available, include a sterilization chamber, which is typically sized to contain a plurality of articles to be sterilized, and equipped with a means of evacuating air and/or other gases from the chamber and adding a sterilant to the chamber. The sterilization process indicator can be placed adjacent an article to be sterilized when placed in the sterilization chamber.

The method includes exposing the sterilization process indicator to a sterilant. The sterilant can be added to the chamber after evacuating the chamber of at least a portion of any air or other gas present in the chamber. Alternatively, sterilant may be added to the chamber without evacuating the chamber. A series of evacuation steps is often used to assure that sterilant reaches all areas within the chamber and contacts all areas of the article(s) to be sterilized. When the sterilant is added to the chamber, the sterilant also contacts the spores under conditions where the sterilant reaches all areas within the chamber.

The method also includes combining the plurality of sterilization resistant spores and the germination medium, thereby bringing the spores and germination medium into contact with each other. This may be done after the sterilization process is completed, that is, after conditions have been provided for the sterilant to reach all areas within the chamber for a time and at a temperature, believed to be sufficient to kill any microorganisms present within the chamber. The medium and the spores can be combined and the combination incubated, both as described above. The detectable change, such as a change in fluorescence, can be monitored and measured continuously or intermittently while incubating the spores with the germination medium. In one alternative, a portion or all of the incubating step may be carried out prior to measuring the detectable change. In another alternative, incubating may be carried out at one temperature, for example, at 50-60° C., and measuring the detectable change may be carried out at a different temperature, for example, at room temperature or at 37° C.

The method also includes measuring the detectable change, if present, in at least one of the one or more dye groups attached to a peptide included in the labeled protease substrate. Viable spores, if present, on contact with the medium and incubation conditions, quickly activate the protease contained by the spores, which cleaves the peptide. The resulting detectable change in a dye group can be measured by measuring an absorbance at a particular wavelength, fluorescence intensity at a particular wavelength, visually assessing a change in color, or the like. Such measurements can be conveniently carried out using known instruments such as a fluorometer, luminometer, spectrophotometer, colorimeter, or the like. For certain embodiments, preferably the detectable change is measured by measuring fluorescence intensity at a particular wavelength.

For certain embodiments, including any one of the above method embodiments, the method further comprising determining whether or not viable spores are present, after exposing the sterilization process indicator to a sterilant, by measuring the detectable change, if present, brought about after incubating the spores with the germination medium as compared with before incubating the spores with the germination medium. For example, fluorescence intensity of the combination of spores and germination medium immediately after combining may serve as a baseline fluorescence. A fluorescence intensity which is greater than the baseline fluorescence after incubating the combination may indicate that viable spores are present. For certain embodiments, fluorescence intensity which is at least 10 percent, preferably, at least 5 percent greater than the baseline indicates that viable spores are present. For certain of these embodiments, whether or not as few as 100 viable spores are present is determined, and wherein the incubating is carried out for not more than 8 hours. For certain of these embodiments, preferably the incubating is carried out for not more than 1 hour. For certain of these embodiments, more preferably, the incubating is carried out for not more than 30 minutes. For certain of these embodiments, even more preferably, the incubating is carried out for not more than 15 minutes.

Alternatively, for certain embodiments, the method further comprises determining whether or not viable spores are present, after exposing the sterilization process indicator to a sterilant, by measuring a rate of the detectable change, if present, such as a rate at which fluorescence intensity changes. For example, after combining the spores with the germination medium and starting the incubating step, the detectable change may be measured continuously or intermittently during the incubation time and the rate of the detectable change determined. For certain embodiments, sterilization resistant spore germination may be detected as a rate of signal increase during the incubation time. This rate may be linear, exponential, or the like, with the respect to incubation time. The rate constant can be used as an indicator of germination of the spores.

Alternatively, for certain embodiments, the method further comprises determining whether or not viable spores are present, after exposing the sterilization process indicator to a sterilant, by taking a final measurement of the detectable change at a specified time. If present, the detectable change, for example a fluorescence intensity, would be higher, for example at least 5 to 10 percent higher, than baseline, for example, baseline fluorescence, previously established for the product. For example, after combining the spores with the germination medium and starting the incubating step, the detectable change, such as fluorescence, may be measured at the end of incubation. The final fluorescence measurement can be used as an indicator of germination of the spores.

The present method, which uses the compositions and indicators described above, can, therefore, be sufficiently sensitive to the presence of viable spores to provide an indication thereof in a short period of time. In addition, the indication can be provided even when the number of viable spores present is relatively low.

For certain embodiments, including any one of the above method embodiments, the method further comprises positioning an article to be sterilized along with the sterilization process indicator in the sterilization chamber. For certain of these embodiments, the method further comprises determining whether or not the sterilization process was effective for sterilizing the article. An indication of no viable spores may be used to determine that the sterilization process was effective for sterilizing the article, whereas an indication of viable spores may be used to determine that the process was not effective. Thus, an assessment of the sterility of an article subjected to a sterilization process may be made in a relatively short time using the composition, indicator, and method embodiments described above.

### Exemplary Embodiments

1. A sterility indicating composition comprising:

a plurality of sterilization process resistant spores which contain an active protease during germination and initial outgrowth of the spores;

a germination medium comprising at least one labeled protease substrate and at least one nutrient for germination of the spores;

wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and

wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores.
2. The composition of embodiment 1, wherein the plurality of sterilization process resistant spores is selected from the group consisting of Gb. stearothermophilus, B. atrophaeus, B. megaterium, Clostridium sporogenes, B. coagulans, and a combination thereof.
3. The composition of embodiment 1 or embodiment 2, wherein the active protease has no more than a background level of activity when subjected to a sterilization process which is just sufficient to decrease a population of at least 1×10⁵ spores to zero, as measured by lack of outgrowth of the spores; and

has a level of activity greater than the background level of activity when subjected to a sterilization process sufficient to decrease the population of at least 1×10⁵ spores by at least one log but to a population greater than zero;

wherein the level of activity is measured by
reacting an effective amount of the at least one labeled protease substrate with the active protease to produce the detectable change in at least one of the one or more dye groups, and
measuring the detectable change.
4. The composition of any one of embodiments 1, 2, and 3, wherein the active protease is germination specific protease.
5. The composition of any one of embodiments 1 through 4, wherein labeled protease substrate is stable at a temperature of at least 60° C. for incubating the spore.
6. The composition of any one of embodiments 1 through 5, wherein the labeled protease substrate is stable at a sterilization temperature.
7. The composition of any one of embodiment s 1 through 6, wherein the labeled protease substrate is stable at a temperature up to at least 121° C.
8. The composition of embodiment 7, wherein the labeled protease substrate is stable at a temperature up to at least 132° C.
9. The composition of any one of embodiments 1 through 8, wherein the labeled protease substrate is a labeled protein, wherein the protein is cleaved by the active protease.
10. The composition of embodiment 9, wherein the labeled protease substrate is selected from the group consisting of a labeled casein, a labeled collagen, a labeled gelatin, a labeled fibrinogen, and a combination thereof, each of which is essentially free of any active protease.
11. The composition of embodiment 10, wherein the labeled protease substrate is a labeled casein.
12. The composition of any one of embodiments 1 through 11, wherein the peptide contains an amino acid sequence selected from the group consisting of AA₁-Glu-AA₂-Ala-AA₃-Glu-Phe, AA₄-Glu-Phe-AA₅-AA₆-Glu-AA₇, and a combination thereof; wherein AA₁ is Tyr, Leu, Phe, or Glu; AA₂ is Ile or Val; AA₃ is Ser, Gln, or Asn; AA₄ is Thr, Ala, Glu, or Gln; AA₅ is Ala, Gly, or Ser; AA₆ is Ser, Thr, or Asn; and AA₇ is Thr or Phe.
13. The composition of any one of embodiments 1 through 11, wherein the detectable change is a change in fluorescence intensity.
14. The composition of embodiment 13, wherein the fluorescence has a wavelength of 550 to 600 nm.
15. The composition of any one of embodiments 1 through 14, wherein the plurality of sterilization process resistant spores and the germination medium are separate from each other and adjacent each other.
16. The composition of any one of embodiments 1 through 15, wherein the germination medium is an aqueous solution or suspension.
17. The composition of any one of embodiments 1 through 15, wherein the germination medium is in a dry form.
18. A sterilization process indicator comprising:

a carrier supporting a plurality of sterilization process resistant spores which contain an active protease during germination and initial outgrowth of the spores;

a container impermeable to microorganisms and impermeable to a sterilant, the container containing a germination medium comprising at least one labeled protease substrate and at least one nutrient for germination of the spores;

wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and

wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and

wherein the carrier is adjacent to the container and separate from the germination medium.
19. The indicator of embodiment 18, wherein the plurality of sterilization process resistant spores is selected from the group consisting of Gb. stearothermophilus, B. atrophaeus, B. megaterium, Clostridium sporogenes, B. coagulans, and a combination thereof.
20. The indicator of embodiment 18 or embodiment 19, wherein the active protease has no more than a background level of activity when subjected to a sterilization process which is just sufficient to decrease a population of at least 1×10⁵ spores to zero, as measured by lack of outgrowth of the spores; and

has a level of activity greater than the background level of activity when subjected to a sterilization process sufficient to decrease the population of at least 1×10⁵ spores by at least one log but to a population greater than zero;

wherein the level of activity is measured by
reacting an effective amount of the at least one labeled protease substrate with the active protease to produce the detectable change in at least one of the one or more dye groups, and
measuring the detectable change.
21. The indicator of any one of embodiments 18, 19, and 20, wherein the active protease is germination specific protease.
22. The composition of any one of embodiments 18 through 21, wherein labeled protease substrate is stable at a temperature of at least 60° C. for incubating the spore.
23. The indicator of any one of embodiments 18 through 22, wherein the labeled protease substrate is stable at a sterilization temperature.
24. The indicator of any one of embodiments 18 through 23, wherein the labeled protease substrate is stable at a temperature up to at least 121° C.
25. The indicator of embodiment 24, wherein the labeled protease substrate is stable at a temperature up to at least 132° C.
26. The indicator of any one of embodiments 18 through 25, wherein the labeled protease substrate is a labeled protein, wherein the protein is cleaved by the active protease.
27. The indicator of embodiment 26, wherein the labeled protease substrate is selected from the group consisting of a labeled casein, a labeled collagen, a labeled gelatin, a labeled fibrinogen, and a combination thereof, each of which is essentially free of any active protease.
28. The indicator of embodiment 27, wherein the labeled protease substrate is a labeled casein.
29. The indicator of any one of embodiments 18 through 28, wherein the peptide contains an amino acid sequence selected from the group consisting of AA₁-Glu-AA₂-Ala-AA₃-Glu-Phe, AA₄-Glu-Phe-AA₅-AA₆-Glu-AA₇, and a combination thereof; wherein AA₁ is Tyr, Leu, Phe, or Glu; AA₂ is Ile or Val; AA₃ is Ser, Gln, or Asn; AA₄ is Thr, Ala, Glu, or Gln; AA₅ is Ala, Gly, or Ser; AA₆ is Ser, Thr, or Asn; and AA₇ is Thr or Phe.
30. The indicator of any one of embodiments 18 through 29, wherein the detectable change is a change in fluorescence intensity.
31. The indicator of embodiment 30, wherein the fluorescence has a wavelength of 550 to 600 nm.
32. The indicator of any one of embodiments 18 through 31, wherein the germination medium is an aqueous solution or suspension.
33. The indicator of any one of embodiments 18 through 31, wherein the germination medium is in a dry form.
34. A method of determining the effectiveness of a sterilization process, the method comprising:

providing a sterilization process indicator comprising:
a carrier supporting a plurality of sterilization process resistant spores which contain an active protease during germination and initial outgrowth of the spores;
a container impermeable to microorganisms and impermeable to a sterilant, the container containing a germination medium comprising at least one labeled protease substrate and at least one nutrient for germination of the spores;
   wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and
   wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and
wherein the carrier is adjacent to the container and separate from the germination medium;

positioning the sterilization process indicator in a sterilization chamber;

exposing the sterilization process indicator to a sterilant;

combining the plurality of sterilization process resistant spores and the germination medium;

incubating the spores with the germination medium;

and

measuring the detectable change, if present.
35. The method of embodiment 34, further comprising determining whether or not viable spores are present, after exposing the sterilization process indicator to a sterilant, by measuring the detectable change, if present, brought about after incubating the spores with the germination medium as compared with before incubating the spores with the germination medium.
36. The method of embodiment 34, further comprising determining whether or not viable spores are present, after exposing the sterilization process indicator to a sterilant, by measuring a rate of the detectable change if present, brought about after incubating the spores with the germination medium as compared with before incubating the spores with the germination medium.
37. The method of embodiment 35 or embodiment 36, wherein whether or not as few as 100 viable spores are present is determined, and wherein incubating the spores is carried out for not more than 8 hours.
38. The method of embodiment 37, wherein incubating the spores is carried out for not more than 1 hour.
39. The method of embodiment 38, wherein incubating the spores is carried out for not more than 30 minutes.
40. The method of any one of embodiments 34 through 39, wherein incubating the spores is carried out at a temperature of at least 60° C.
41. The method of any one of embodiments 34 through 40, further comprising positioning an article to be sterilized along with the sterilization process indicator in the sterilization chamber.
42. The method of embodiment 41, further comprising determining whether or not the sterilization process was effective for sterilizing the article.
43. The method of any one of embodiments 40 through 42, wherein the plurality of sterilization process resistant spores is selected from the group consisting of Gb. stearothermophilus, B. atrophaeus, B. megaterium, Clostridium sporogenes, B. coagulans, and a combination thereof.
44. The method of any one of embodiments 34 through 43, wherein the active protease

has no more than a background level of activity when subjected to a sterilization process which is just sufficient to decrease a population of at least 1×10⁵ spores to zero, as measured by lack of outgrowth of the spores; and

has a level of activity greater than the background level of activity when subjected to a sterilization process sufficient to decrease the population of at least 1×10⁵ spores by at least one log but to a population greater than zero;

wherein the level of activity is measured by
reacting an effective amount of the at least one labeled protease substrate with the active protease to produce the detectable change in at least one of the one or more dye groups, and
measuring the detectable change.
45. The method of any one of embodiments 34 through 44, wherein the active protease is a germination specific protease.
46. The method of any one of embodiments 34 through 45, wherein labeled protease substrate is stable at a temperature of at least 60° C. for incubating the spore.
47. The method of any one of embodiments 34 through 46, wherein the labeled protease substrate is stable at a sterilization temperature.
48. The method of any one of embodiments 34 through 47, wherein the labeled protease substrate is stable at a temperature up to at least 121° C.
49. The method of embodiment 48, wherein the labeled protease substrate is stable at a temperature up to at least 132° C.
50. The method of any one of embodiments 34 through 48, wherein the labeled protease substrate is a labeled protein, wherein the protein is cleaved by the by the active protease.
51. The method of embodiment 50, wherein the labeled protease substrate is selected from the group consisting of a labeled casein, a labeled collagen, a labeled gelatin, a labeled fibrinogen, and a combination thereof, each of which is essentially free of any active protease.
52. The method of embodiment 51, wherein the labeled protease substrate is a labeled casein.
53. The method of any one of embodiments 34 through 52, wherein the peptide contains an amino acid sequence selected from the group consisting of AA₁-Glu-AA₂-Ala-AA₃-Glu-Phe, AA₄-Glu-Phe-AA₅-AA₆-Glu-AA₇, and a combination thereof; wherein AA₁ is Tyr, Leu, Phe, or Glu; AA₂ is Ile or Val; AA₃ is Ser, Gln, or Asn; AA₄ is Thr, Ala, Glu, or Gln; AA₅ is Ala, Gly, or Ser; AA₆ is Ser, Thr, or Asn; and AA₇ is Thr or Phe.
54. The method of any one of embodiments 34 through 53, wherein the detectable change is a change in fluorescence intensity.
55. The method of embodiment 44, wherein the fluorescence has a wavelength of 550 to 600 nm.
56. The method of any one of embodiments 34 through 55, wherein the plurality of sterilization process resistant spores and the germination medium are separate from each other and adjacent each other.
57. The method of any one of embodiments 34 through 56, wherein the germination medium is an aqueous solution or suspension.
58. The method of any one of embodiments 34 through 56, wherein the germination medium is in a dry form.

Objects and advantages of this invention are further illustrated by the following examples, but the particular materials and amounts thereof recited in these examples, as well as other conditions and details, should not be construed to unduly limit this invention.

### EXAMPLES

### Spore Suspensions

Spore suspensions of Gb. stearothermophilus (also known as Bacillus stearothermophilus) were prepared by known methods as described in Example 1 of U.S. Pat. No. 5,418,167 (Matner et al.). Modifications of these methods and alternative methods known to those skilled in the art may also be used for preparing these suspensions.

### Example 1

### Protease Enzyme Detection

Dilutions of a Gb. stearothermophilus spore suspension were performed in sterile water for irrigation (Baxter, Deerfield, Ill.). The resulting diluted spore preparations (1 μL) were tested at a final population of 1×10⁶, 1×10⁵, 1×10⁴, 1×10³ and 0 spores in a plate reader as follows. The spores were allowed to dry for 20 min in a 37° C. incubator. The spores were then rehydrated in 100 uL of medium, consisting of 50 uL GFK (1 mg/mL glucose, 1 mg/mL fructose, 3.3 mg/mL potassium chloride), 25 uL of Tris buffer with 0.2 mM CaCl, and 25 uL of 4 mg/mL labeled protease substrate (Calbiochem, Casein labeled with N-(resorufin-4-carbonyl)piperidine-4-carbonic acid, Protease substrate (EMD Chemicals, Gibbstown, N.J.). Each resulting mixture was added to a well of a plate. The plate was subsequently placed in a preheated Synergy 4 plate reader (BioTech, Winooski, Vt.) at 50° C. and incubated for 480 minutes. During this time, fluorescence intensity readings at an emission wavelength of 580 nm after excitation at 520 nm were taken at intervals for a total of 96 readings for each well. A summary of the results is shown in Table 1.

TABLE 1
Fluorescence Intensity vs. Time at Various Spore Populations

| Time (hrs) | 0 | 1.0E+03 | 1.0E+04 | 1.0E+05 | 1.0E+06 |
|---|---|---|---|---|---|
| 0 | 0 | 0 | 0 | 0 | 0 |
| 0.5 | −2518.5 | −5425 | −2860 | 30764.5 | 66762 |
| 1 | −3106.5 | −1542.5 | 9260.5 | 65782.5 | 66762 |
| 4 | −2292 | 4999.5 | 53773 | 71862.5 | 66762 |
| 8 | 471.5 | 25551.5 | 62521.5 | 71862.5 | 66762 |

The data was normalized to the initial time zero reading by subtracting the initial time zero reading from all readings. The results illustrate, for example, the detection of active protease enzyme and a correlation of this to the number of viable spores present in the sample.

### Example 2

### Stability of Labeled Protease Substrate to Exposure to Sterilization Temperatures

Dilutions of a Gb. stearothermophilus spore suspension were performed in sterile water for irrigation (Baxter, Deerfield, Ill.). The resulting diluted spore preparations (1 μL) were tested at a final population of 1×10⁶, and 0 spores. The spores were allowed to dry for 20 min in a 37° C. incubator. The spores were then rehydrated in 100 uL of medium, consisting of 50 uL GFK (1 mg/mL glucose, 1 mg/mL fructose, 3.3 mg/mL potassium chloride), 25 uL of Tris buffer with 0.2 mM CaCl, and 25 uL of 4 mg/mL labeled protease substrate as in Example 1, that was previously run through a 15 min 121° C. (250° F.) vacuum assisted cycle in a AMSCO Scientific SG-120 Eagle/Century Series steam sterilizer (Steris, Mentor, Ohio). A medium that was not subjected to the sterilization conditions (un-autoclaved protease medium) was tested simultaneously as a positive control. Each resulting mixture was placed in a well of a plate, and the plate was subsequently placed in a preheated Synergy 4 plate reader (BioTech, Winooski, Vt.) at 50° C. and incubated for 180 min. During this time, fluorescence intensity readings at an emission wavelength of 580 nm after excitation at 520 nm were taken at intervals for a total of 36 readings for each well. A summary of the results is shown in Table 2.

TABLE 2
Fluorescence Intensity vs. Time at a Spore Population
of 0 and 10⁶ With and Without Autoclaving the Medium

| | |
|---|---|
| Autoclaved Protease | Un-autoclaved Protease |
| Medium | Medium |

| Time (hrs) | 0 | E6 | 0 | E6 |
|---|---|---|---|---|
| 0 | 0 | 0 | 0 | 0 |
| 0.5 | 5816 | 21953 | −3416 | 20646 |
| 1 | 2176 | 18119 | −4297 | 21559 |
| 1.5 | 1035 | 21674 | −4480 | 24242 |
| 2 | 424 | 23561 | −4433 | 24099 |
| 2.5 | 34 | 23154 | −4294 | 24031 |
| 3 | −213 | 22910 | −3737 | 23948 |

The data was normalized to the initial time zero reading by subtracting the initial time zero reading from all readings. The results illustrate, for example, that the protease substrate retained its ability to be cleaved by an active protease and generate fluorescence even after being exposed to sterilization processes.

### Example 3

### Protease Activity After Population of at Least 10⁵ Spores is Decreased to Zero by a Sterilization Process

Two glass vials, one containing 1 mL of a Gb. stearothermophilus spore suspension, and the other containing 1 mL of medium with labeled protease substrate was placed in a sterilizer and run in a 15 min 121° C. (250° F.) vacuum assisted cycle in a AMSCO Scientific SG-120 Eagle/Century Series steam sterilizer (Steris, Mentor, Ohio). The resulting sterilized spore crop and medium were subsequently tested for the presence of a protease substrate. Unautoclaved spore suspensions were tested simultaneously as a positive control. The spores were allowed to dry for 20 min in a 37° C. incubator. The spores were then rehydrated in 100 uL of the sterilized medium, consisting of 50 uL GFK (1 mg/mL glucose, 1 mg/mL fructose, 3.3 mg/mL potassium chloride), 25 uL of Tris buffer with 0.2 mM CaCl, and 25 uL of 4 mg/mL labeled protease substrate as in Example 1. The resulting mixtures were each added to a well of a plate. The plate was subsequently placed in a preheated Synergy 4 plate reader (BioTech, Winooski, Vt.) at 50° C. and incubated for 180 min. During this time, fluorescence intensity readings at an emission wavelength of 580 nm after excitation at 520 nm were taken at intervals for a total of 36 readings for each well. A summary of the results is shown in Table 3.

TABLE 3
Fluorescence Intensity vs. Time at Spore Populations of 0 and 10⁵
Un-autoclaved and Autoclaved With Autoclaved Medium

| | | |
|---|---|---|
| | Autoclaved | |
| Time | Spores | Live Spores |

| (min) | 0 | E5 | 0 | E5 |
|---|---|---|---|---|
| 0 | 0 | 0 | 0 | 0 |
| 10 | 274 | 5496 | −218 | 33543 |
| 30 | −1290 | 5,042 | −3416 | 36,682 |
| 60 | −1649 | 2,689 | −4297 | 46,634 |
| 90 | −1551 | 2,136 | −4480 | 55,638 |
| 120 | −1543 | 2,120 | −4433 | 57,447 |
| 180 | −890 | 2,188 | −3737 | 60,416 |

The data was normalized to the initial time zero reading by subtracting the initial time zero reading from all readings. The results illustrated, for example, that dead spores that were a result of a sterilization process did not have detectable levels of protease that could hydrolyze the protease substrate. Live spores on the other hand were capable of producing large amounts of active protease as measured by an increase in fluorescence. Any active protease from the spores had no more than a background level of activity after having been subjected to the sterilization process which was just sufficient to decrease the population of at least 1×10⁵ spores to zero.

### Example 4

### Detection of Active Protease After Exposure of Spores to a Sub-Lethal Sterilization Cycle

Biological indicators were prepared by coating a suspension of Gb. stearothermophilus spores at E5-E6 on a polypropylene carrier. Following drying at 37° C. for 20 min, the biological indicators were exposed to commonly used sterilization conditions: a) 132° C., AMSCO EAGLE Model 2013 steam sterilizer (Steris, Mentor Ohio), b) 132° C. vacuum assisted cycles in a Joslyn Steam Biological Indicator Evaluator Resistometer (BIER) vessel (Steris, Mentor Ohio). Following sterilization, the biological indicators were contacted with medium containing GFK and labeled protease substrate as in Examples 1-3, but with hydroxypyrenetrisulfonic acid at 0.1 mg/ml added, and fluorescence intensity readings at an emission wavelength of 580 nm after excitation at 520 nm were taken at 30 and 60 min intervals at an incubation temperature of 50° C. Fluorescence intensity readings at an emission wavelength of 560 nm after excitation at 530 nm were taken at 30 and 60 min intervals at an incubation temperature of 60° C. using a 96 well sample fluorescence measuring device with temperature control. Summaries of the results are shown in Tables 4-6.

TABLE 4
Change in Fluorescence After 30 Minutes and 60 Minutes at an
Incubation Temperature of 50° C. Subsequent to
Sterilization Condition a) for One and Three Minutes.

| | Change in fluorescence | Change in fluorescence |
|---|---|---|
| Time in sterilizer | intensity at 30 min | intensity at 60 min |
| 1 min | 32,716 | 42,116 |
| 3 min | −319 | 311.4 |
| 0 (Positive control) | 67,751 | 100,429 |
| 0 (Negative control) | −345 | −78.5 |

TABLE 5
Change in Fluorescence After 30 Minutes and 60 Minutes at an
Incubation Temperature of 60° C. Subsequent to
Sterilization Condition a) for One and Three Minutes.

| | Change in fluorescence | Change in fluorescence |
|---|---|---|
| Time in sterilizer | intensity at 30 min | intensity at 60 min |
| 1 min | 3,847 | 5,620 |
| 3 min | −360 | −141 |

TABLE 6
Change in Fluorescence After 30 Minutes and 60 Minutes at an
Incubation Temperature of 50° C. Subsequent to
Sterilization Condition b) for One and Three Minutes.

| | Change in fluorescence | Change in fluorescence |
|---|---|---|
| Time in sterilizer | intensity at 30 min | intensity at 60 min |
| 1 min | 22,248 | 54,285 |
| 3 min | 341 | 815 |
| 0 (Negative control) | −468 | −182 |

In all of the above, the active protease was detected after exposure to sub-lethal sterilization cycles of 1 minute duration. Independent evidence of viable spores in these samples was observed based upon the acid sensitive dye, hydroxypyrenetrisulfonic acid.

### Example 5

### Protease Enzyme Detection as a Biological Indicator Utilizing a Fluorescent Labeled Short Peptide Sequence

A peptide having the following sequence was selected from the germination protease (GPR) cleavage site in Bacillus subtilis small acid soluble spore proteins (SASP) according to Y. Carrillo-Martinez and P. Setlow, J. Bacteriol. 1994 September, 176(17), 5357-5363: Tyr-Glu-Ile-Ala-Ser-Glu-Phe, where the cleavage site is the amide bond between Glu and Ile.

First, the peptide was labeled as follows with one tetramethylrhodamine (TMR) label: TMR-Tyr-Glu-Ile-Ala-Ser-Glu-Phe-Lys-Amide This was carried out using a solid phase peptide synthesis by Genemed Synthesis Inc (San Antonio, Tex.), where a Lys residue was also added for subsequent attachment of a second dye group. The labeled peptide was purified to 99% purity by high performance liquid chromatography (HPLC) and confirmed by matrix-assisted laser desorption/ionization (MALDI) mass spectroscopy.

Subsequently, the above single-labeled peptide was reacted in aqueous solution (100 mM bicarbonate buffer, pH 9) with 5,6-carboxyl tetramethylrhodamine succinimidyl ester (Molecular Probes, Eugene, Oreg.) to make the following doubly labeled peptide: TMR-Tyr-Glu-Ile-Ala-Ser-Glu-Phe-Lys(TMR)-Amide. The resulting reaction mixture was purified by HPLC and fractions were collected for enzymatic cleavage by GPR and fluorescent testing.

Dilutions of a Gb. Stearothermophilus spore preparation were performed in GFK (1 mg/ml glucose, 1 mg/ml fructose, 3.3 mg/ml potassium chloride) to a final population of 2.5×10⁶ and immediately added 50 uL of media, consisting of 45 uL of Tris buffer with 0.2 mM CaCl, and 5 uL of the above mentioned doubly labeled protease substrate in each well. The plate was subsequently placed in a preheated Synergy 4 plate reader at 50° C. (BioTeck Instruments Inc, Winoski, Vt.) and incubated for up to 300 min with fluorescent excitation at 520 nm and emission at 580 nm wavelengths. Selected resulting fluorescence data points in the time-course are summarized as follows.

| Minutes | No Spores | 2.5E+06 Spores |
|---|---|---|
| 0 | 0.0 | 0.0 |
| 30 | 145.0 | 242.0 |
| 45 | 198.0 | 532.5 |
| 60 | 269.0 | 782.5 |
| 90 | 385.0 | 1168.0 |
| 120 | 484.0 | 1442.0 |
| 180 | 568.0 | 1909.5 |
| 240 | 634.0 | 2257.0 |
| 300 | 673.0 | 2443.0 |

These results showed that the detection of protease activity utilizing a fluorescent labeled peptide sequence correlated to the presence of viable cells in the sample.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

The complete disclosures of the patents, patent documents, and publications cited herein are incorporated by reference in their entirety or the portions of each that are indicated as if each were individually incorporated.

1. A sterility indicating composition comprising:
a plurality of sterilization process resistant spores which contain an active protease during germination and initial outgrowth of the spores;
a germination medium comprising at least one labeled protease substrate and at least one nutrient for germination of the spores;
wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and
wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores.

2. The composition of claim 1, wherein the plurality of sterilization process resistant spores is selected from the group consisting of Gb. stearothermophilus, B. atrophaeus, B. megaterium, Clostridium sporogenes, B. coagulans, and a combination thereof.

3. The composition of claim 1, wherein the active protease has no more than a background level of activity when subjected to a sterilization process which is just sufficient to decrease a population of at least 1×10⁵ spores to zero, as measured by lack of outgrowth of the spores; and
has a level of activity greater than the background level of activity when subjected to a sterilization process sufficient to decrease the population of at least 1×10⁵ spores by at least one log but to a population greater than zero;
wherein the level of activity is measured by
reacting an effective amount of the at least one labeled protease substrate with the active protease to produce the detectable change in at least one of the one or more dye groups, and
measuring the detectable change.

4. The composition of claim 1, wherein the active protease is germination specific protease.

5. The composition of claim 1, wherein labeled protease substrate is stable at a temperature of at least 60° C. for incubating the spore.

6. The composition of claim 1, wherein the labeled protease substrate is a labeled protein, wherein the protein is cleaved by the active protease.

7. The composition of claim 6, wherein the labeled protease substrate is selected from the group consisting of a labeled casein, a labeled collagen, a labeled gelatin, a labeled fibrinogen, and a combination thereof, each of which is essentially free of any active protease.

8. The composition of claim 1, wherein the detectable change is a change in fluorescence intensity.

9. A sterilization process indicator comprising:
a carrier supporting a plurality of sterilization process resistant spores which contain an active protease during germination and initial outgrowth of the spores;
a container impermeable to microorganisms and impermeable to a sterilant, the container containing a germination medium comprising at least one labeled protease substrate and at least one nutrient for germination of the spores;
wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and
wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and
wherein the carrier is adjacent to the container and separate from the germination medium.

10. The indicator of claim 9, wherein the plurality of sterilization process resistant spores is selected from the group consisting of Gb. stearothermophilus, B. atrophaeus, B. megaterium, Clostridium sporogenes, B. coagulans, and a combination thereof.

11. The indicator of claim 9, wherein the active protease has no more than a background level of activity when subjected to a sterilization process which is just sufficient to decrease a population of at least 1×10⁵ spores to zero, as measured by lack of outgrowth of the spores; and
has a level of activity greater than the background level of activity when subjected to a sterilization process sufficient to decrease the population of at least 1×10⁵ spores by at least one log but to a population greater than zero;
wherein the level of activity is measured by
reacting an effective amount of the at least one labeled protease substrate with the active protease to produce the detectable change in at least one of the one or more dye groups, and
measuring the detectable change.

12. The indicator of claim 9, wherein the active protease is germination specific protease.

13. The composition of claim 9, wherein labeled protease substrate is stable at a temperature of at least 60° C. for incubating the spore.

14. The indicator of claim 9, wherein the labeled protease substrate is a labeled protein, wherein the protein is cleaved by the active protease.

15. A method of determining the effectiveness of a sterilization process, the method comprising:
providing a sterilization process indicator comprising:
a carrier supporting a plurality of sterilization process resistant spores which contain an active protease during germination and initial outgrowth of the spores;
a container impermeable to microorganisms and impermeable to a sterilant, the container containing a germination medium comprising at least one labeled protease substrate and at least one nutrient for germination of the spores;
wherein the medium is essentially free of a) any active protease other than the active protease contained by the plurality of spores and b) any protease substrate other than the at least one labeled protease substrate, other than any protease substrate originating from the plurality of spores, and other than any protease substrate which does not compete with the labeled protease substrate for the active protease; and
wherein the at least one labeled protease substrate comprises a peptide which can be cleaved by the active protease and which is labeled with one or more dye groups, at least one of which undergoes a detectable change when the peptide is cleaved by the active protease, and wherein the labeled protease substrate is stable at least at a temperature for incubating the spores; and
wherein the carrier is adjacent to the container and separate from the germination medium;
positioning the sterilization process indicator in a sterilization chamber;
exposing the sterilization process indicator to a sterilant;
combining the plurality of sterilization process resistant spores and the germination medium;
incubating the spores with the germination medium;
and
measuring the detectable change, if present.

16. The method of claim 15, further comprising determining whether or not viable spores are present, after exposing the sterilization process indicator to a sterilant, by measuring the detectable change, if present, brought about after incubating the spores with the germination medium as compared with before incubating the spores with the germination medium.

17. The method of claim 15, further comprising determining whether or not viable spores are present, after exposing the sterilization process indicator to a sterilant, by measuring a rate of the detectable change if present, brought about after incubating the spores with the germination medium as compared with before incubating the spores with the germination medium.

18. The method of claim 16, wherein whether or not as few as 100 viable spores are present is determined, and wherein incubating the spores is carried out for not more than 8 hours.

19. The method of claim 15, further comprising positioning an article to be sterilized along with the sterilization process indicator in the sterilization chamber.

20. The method of claim 19, further comprising determining whether or not the sterilization process was effective for sterilizing the article.
